(19) United States
(12) Patent Application Publication
(10) Pub. No.: US 2011/0021745 A1
(43) Pub. Date: Jan. 27, 2011

(54) ULTRA-SMALL CHITOSAN NANOPARTICLES USEFUL AS BIOIMAGING AGENTS AND METHODS OF MAKING SAME

(76) Inventors: Swadeshmukul Santra, Orlando, FL (US); Padmavathy Tallury, Orlando, FL (US)

Correspondence Address: ALLEN, DYER, DOPPELT, MILBRATH & GILCHRIST, P.A., 255 SOUTH ORANGE AVENUE, STE. 1401, ORLANDO, FL, 32801, US

(21) Appl. No.: 12/667,711

(22) PCT Filed: Jul. 7, 2008
(86) PCT No.: PCT/US08/69299

§ 371 Date: Oct. 7, 2010

Related U.S. Application Data
(60) Provisional application No. 60/948,203, filed on Jul. 6, 2007.

(51) Int. Cl. C07K 2/00; C08B 37/08; C07K 16/00

(52) U.S. Cl. 530/322; 536/20; 530/391.1; 977/773; 977/774; 977/795; 977/906; 977/927; 977/930

(57) ABSTRACT

A method of making ultra-small chitosan nanoparticles having a size range of approximately 10-20 nm, includes preparing a first microemulsion containing effective amounts of cyclohexane, n-hexanol, chitosan polymer and a nonionic surfactant. A second microemulsion is prepared containing effective amounts of cyclohexane, n-hexanol, tartaric acid, EDC, n-hydroxysuccinimide, and a nonionic surfactant. The method continues by reacting the first and second microemulsions for a time sufficient to form the ultra-small chitosan nanoparticles and recovering the nanoparticles from the reacted microemulsion. The chitosan polymer may be crosslinked and may also be tagged with a fluorescent compound, a radio-opaque compound, a paramagnetic ion, a ligand specific for a predetermined biologic target, a drug, and combinations thereof.

### RELATED APPLICATION

This application claims priority from co-pending provisional application Ser. No. 60/948,203, which was filed on 6 Jul. 2007, and which is incorporated herein by reference in its entirety.

### STATEMENT OF GOVERNMENT RIGHTS

The work leading to this invention was partly supported by a grant from the National Science Foundation. Accordingly, the government may have certain rights in the invention, as specified by law.

### FIELD OF THE INVENTION

The present invention relates to the field of biomedical imaging and, more particularly, to ultra-small nanoparticles of chitosan useful as bioimaging agents and to methods for making such nanoparticles.

### BACKGROUND OF THE INVENTION

The rapidly growing field of biomedical imaging has made substantial contribution in today's healthcare system. This has enabled many advanced diagnostic procedures that are based upon visualization of physiological structures, measurement of biological functions, and evaluation of cellular and molecular events without requiring invasive procedures. In spite of these advancements in diagnostic imaging capabilities, especially with the modalities of ultrasound, nuclear medicine, nuclear magnetic resonance and spectroscopy, X-ray/CT, optical, endoscopic, and visualization strategies, the hard reality is that millions of Americans die in the United States each year due to poor and late diagnosis. This urgently demands a revolutionary breakthrough in diagnostic biomedical imaging.

To save countless lives each year, better imaging technologies (i.e. imaging system as well as image contrast agents) must be developed that are highly sensitive, non-invasive, accurate and suitable for early diagnosis. Further technology development is also necessary for real-time multimodal imaging applications.

In routine diagnostic imaging procedure, “blood-pool” contrast media are used to boost signal-to-noise ratio that provides better image contrast. These contrast media are non-targeted, single-modal and designed for single imaging application, requiring a high-dose of such contrast agents to obtain significant contrast between the target tissue and the normal tissue. There is a great demand for developing high resolution target specific contrast agents with real-time multimodal imaging capabilities.

For accurate diagnosis, surgeons often recommend to patients multiple diagnostic imaging procedures. For many decades, patients have been moved from one imaging machine to another to obtain data from different imaging modalities. Surgeons heavily rely upon processed data that are obtained by fusing images from different modalities using image-fusion software. Artifacts in such processed data are evident especially when (i) image contrast (resolution) is poor, (ii) target is displaced when the patient moved from one scanner to another scanner and (iii) coordinates for the reconstruction of three-dimensional (3D) multimodal images using the image-fusion software are not accurately defined. The use of a suitable multimodal contrast agent will have the capability to get rid of all these artifacts.

The future of next generation (multimodal) in vivo imaging systems: Future advancements certainly lie in the directions of new and improved imaging modalities. Since no single imaging modality can provide information on all aspects of structure and function, an obvious approach is to interrogate a subject using multiple imaging modalities. Multimodal imaging systems are capable of providing important scientific or diagnostic information not readily attainable using two separate imaging systems, and where possible, the performance of each imaging system will remain preserved. Multimodal imaging systems are being developed for clinical applications (e.g., diagnostics and for clinical trials of new therapeutics) and for preclinical applications (e.g. drug development, evaluating cell and gene-based therapies, and new molecular imaging assays). The combination of structural and functional/molecular imaging techniques, especially PET/CT and SPECT/CT, is the most successful example of multimodality imaging systems to date.

### SUMMARY OF THE INVENTION

With the foregoing in mind, the present invention advantageously provides a method of making ultra-small chitosan nanoparticles. The method comprises preparing a first microemulsion containing effective amounts of cyclohexane, n-hexanol, chitosan polymer and a nonionic surfactant. A second microemulsion is then prepared, containing effective amounts of cyclohexane, n-hexanol, tartaric acid. EDC, n-hydroxysuccinimide, and a nonionic surfactant. The method continues by reacting the first and second microemulsions for a time sufficient to form the ultra-small chitosan nanoparticles, then recovering the nanoparticles from the reacted microemulsion.

The chitosan polymer may be modified in the method. For example, one variation of the method calls for the chitosan polymer to be covalently crosslinked by reacting with a dicarboxylic acid in a water-in-oil microemulsion. Moreover, the chitosan polymer may comprise a proportion of the polymer linked to a succinic acid functional group so that recovered nanoparticles are formed by non-crosslinked electrostatically held chitosan and succinic anhydride chitosan.

In the method of the invention, the nonionic surfactant preferably comprises Triton X-100 and the tartaric acid is in an aqueous solution. Also, in the reacting step the mixing conditions are at room temperature. Both microemulsions (ME) are individually prepared under magnetically stirred conditions and ME-II is added drop-wise to a magnetically stirred ME-I. After the addition is finished, the microemulsions are continuously mixed by stirring for 24 hours to ensure a complete reaction. Dark conditions are maintained only for experiments that involve fluorescein isothiocyanate (FITC) or iohexyl, otherwise normal room light conditions are maintained during stirring. Recovering of the particles after reacting is effected by addition of ethanol so as to separate the nanoparticles from the microemulsion. Addition of the ethanol destabilizes the microemulsion system resulting in the precipitation of the nanoparticles from the microemulsion. Use of 95% (V/V) ethanol for this application is preferred.

After reacting and recovering the method further comprises washing the recovered nanoparticles in ethanol at least once, followed by suspending the recovered nanoparticles in a fluid carrier, preferably in water. In order to further clean the particle suspension, the suspended recovered nanoparticles are dialysed against water. In washing, the nanoparticles are pelleted by centrifugation at 8000 rpm in an Eppendorf, model 5810R, angle-head centrifuge, in a 35 ml total volume for 15 mins. Those skilled in the art will be able to determine centrifugation conditions necessary for pelleting these nanoparticles in other centrifuge systems. In washing, ethanol was added to the centrifuged nanoparticles followed by vortexing for a few minutes and then sonication (using a sonic bath) for about 10 seconds. This allowed nanoparticles to re-disperse uniformly in the ethanol. This ethanol solution was then centrifuged for 15 minutes. Nanoparticles at this stage settled down at the bottom of the centrifuge tube. The supernatant was then discarded. This washing procedure (addition of ethanol to the centrifuged nanoparticles, vortexing the solution followed by sonication, centrifugation and removal of the supernatant) was repeated for 5 times. Washed nanoparticles are resuspended in a fluid carrier, preferably water, and aggregated nanoparticles are separated from monodispersed nanoparticles by filtration.

In other embodiments of the method, the chitosan polymer is further covalently labeled with fluorescein isothiocyanate so that the recovered nanoparticles exhibit fluorescence. Alternatively, the chitosan polymer may be linked to a sequestering agent having an MRI (magnetic resonance imaging) contrast agent bound therein so that the recovered nanoparticles are effective as an MRI contrast medium. The MRI contrast agent comprises a paramagnetic ion selected from gadolinium, dysprosium, europium, and compounds and combinations thereof. Furthermore, the chitosan polymer may be linked with iohexyl so that the nanoparticles are radio-opaque.

The method may be modified where the chitosan polymer comprises a mixture of fluorescein isothiocyanate-labeled chitosan and chitosan linked with a sequestering agent having a paramagnetic chelate bound therein so that the recovered nanoparticles are effective as a bimodal agent which is fluorescent as well as paramagnetic.

Similarly, in another embodiment, the method calls for the chitosan polymer to comprise a mixture of fluorescein isothiocyanate-labeled chitosan and chitosan polymer linked with iohexyl so that the recovered nanoparticles are effective as a bimodal agent which is fluorescent as well as radio-opaque.

In yet another embodiment of the method the chitosan polymer is conjugated with a ligand for a predetermined biological target so that recovered nanoparticles are effective as target-specific probes. The ligand is preferably selected from a peptide, an oligonucleotide, folic acid, an antigen, an antibody, and combinations thereof. Instead of the ligand, the chitosan polymer may be conjugated with a drug.

The present invention includes the various chitosan nanoparticles made by the methods disclosed. For example, nanoparticles comprising chitosan polymer, having a range of from approximately 10 to 20 nm in size and having a zeta potential of approximately 22 to 33 mV. In these nanoparticles, the chitosan polymer may be covalently crosslinked. In other nanoparticles, the chitosan polymer comprises a proportion of the polymer linked to a succinic acid functional group so that recovered nanoparticles are formed by non-crosslinked electrostatically held chitosan and succinic anhydride chitosan.

Nanoparticles according to the invention include those wherein the chitosan polymer is covalently labeled with fluorescein isothiocyanate so that the nanoparticles exhibit fluorescence. In another embodiment of the nanoparticles the chitosan polymer is linked to a sequestering agent having an MRI contrast agent bound therein so that the nanoparticles are effective as an MRI contrast medium. When the chitosan polymer is linked with iohexyl the nanoparticles are radio-opaque.

Multimodal nanoparticles are included within the scope of the invention. For example, nanoparticles wherein the chitosan polymer comprises a mixture of fluorescein isothiocyanate-labeled chitosan and chitosan linked with a sequestering agent having a paramagnetic chelate bound therein so that the nanoparticles are effective as a bimodal agent which is fluorescent as well as paramagnetic. The chitosan polymer may also comprise a mixture of fluorescein isothiocyanate-labeled chitosan and chitosan polymer linked with iohexyl so that the recovered nanoparticles are effective as a bimodal agent which is fluorescent as well as radio-opaque.

The nanoparticles of the present invention may be employed as biologic agents in that, for example, the chitosan polymer may be conjugated with a ligand for a predetermined biological target so that nanoparticles are effective as target-specific probes. Likewise, the chitosan polymer may be conjugated with a biologically active drug. When these two modalities are combined, the disclosed nanoparticles are useful as target-specific drug delivery vehicles.

### BRIEF DESCRIPTION OF THE DRAWINGS

Some of the features, advantages, and benefits of the present invention having been stated, others will become apparent as the description proceeds when taken in conjunction with the accompanying drawings, presented for solely for exemplary purposes and not with intent to limit the invention thereto, and in which:

FIG. 1 is a schematic representation of the preparation of chitosan nanoparticles by the water-in-oil micromemulsion technique according to an embodiment of the present invention;

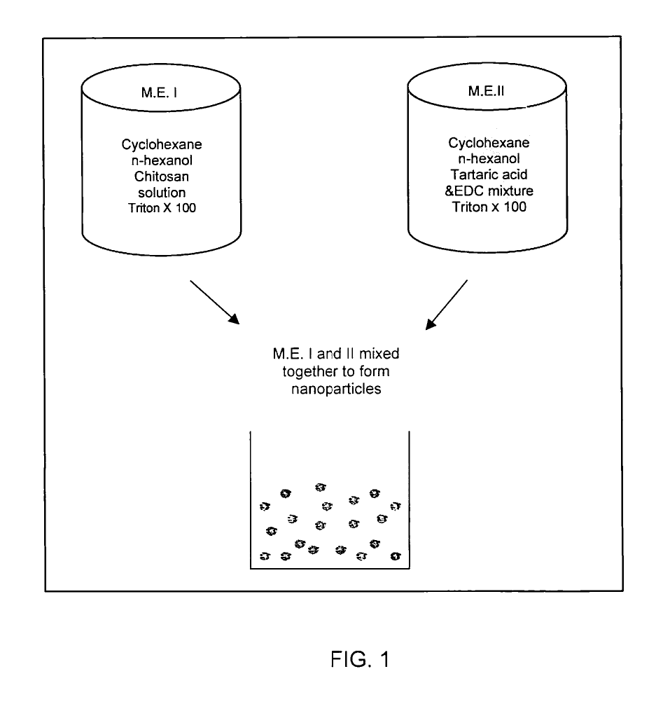

FIG. 2 shows a TEM image of ultra-small chitosan nanoparticles prepared from a 0.25% chitosan solution, as described below;

FIG. 3 graphically displays particle size distribution of nanoparticles prepared from 0.25% chitosan solution;

FIG. 4 depicts excitation and emission spectra of FITC moiety in the FITC labeled chitosan nanoparticles;

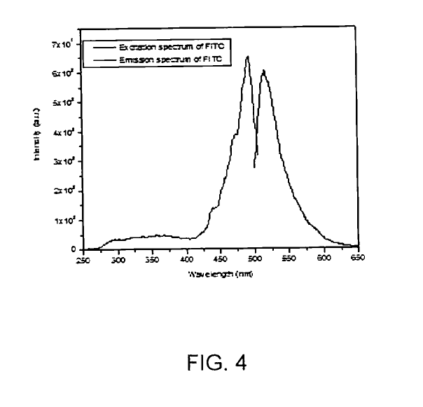

FIG. 5 provides a TEM image of FITC labeled chitosan particles prepared from 0.25% chitosan;

FIG. 6 is a TEM image of FITC labeled chitosan particles prepared from 0.50% chitosan;

FIG. 7 are digital images of FITC labeled ˜15 nm size chitosan nanoparticles (concentration 1.0 mg/ml) dispersed in DI water; (a) day light image and (b) fluorescence image taken under a hand held 366 nm multiband excitation source;

FIG. 8 Magnetic resonance image of paramagnetic chitosan nanoparticles prepared from 0.25% chitosan under a MRI scanner of 4.5 T; C1 is the initial concentration of the nanoparticles that shows a bright image and has a relaxation time T1 of 257.26 ms; C2 to C5 are the diluted concentrations of the nanoparticle solution; as the concentration of the nanoparticle solution is decreased from C2 to C5, the brightness of the images decreases and the image brightness is equal to that of water;

FIG. 9 shows a TEM image of folate and FITC conjugated chitosan nanoparticles;

FIG. 10 is a graph showing excitation and emission spectra of folate and FITC labeled chitosan nanoparticles; and

FIG. 11 depicts the excitation and emission spectra of folate and FITC labeled chitosan nanoparticles.

### DETAILED DESCRIPTION OF THE PREFERRED EMBODIMENT

The present invention will now be described more fully hereinafter with reference to the accompanying drawings, in which preferred embodiments of the invention are shown. Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. Any publications, patent applications, patents, or other references mentioned herein are incorporated by reference in their entirety. In case of conflict, the present specification, including any definitions, will control. In addition, the materials, methods and examples given are illustrative in nature only and not intended to be limiting. Accordingly, this invention may, however, be embodied in many different forms and should not be construed as limited to the illustrated embodiments set forth herein. Rather, these illustrated embodiments are provided so that this disclosure will be thorough and complete, and will fully convey the scope of the invention to those skilled in the art. Other features and advantages of the invention will be apparent from the following detailed description, and from the claims.

### Materials and Methods

Chitosan (molecular weight 50,000-190,000 daltons), Triton X 100, 1-ethyl-3-(3-dimethyl aminopropyl carbodiimide) (EDC), hydroxysuccinimide were purchased from Sigma Chemical company. Cyclohexane, n-hexanol, ethanol, fluorescein isothiocyanate (FITC), folic acid, gadolinium acetate dehydrate, EDTA were purchased from Fisher scientific company. DOTA-NHS was procured from Macrocyclics. DOTA-NHS is an amine reactive Gadolinium ion (Gd3+) chelator; it is clinically used under the name “Gadoteridol”.

### Characterization Techniques

The size of the nanoparticles was determined by Malvern Zeta sizer dynamic light scattering (DLS) instrument and transmission electron microscopy (TEM) (JEOL, JEM 1011 100 kV). The surface charge (zeta potential) of the nanoparticles was determined by Malvern Zeta sizer Dynamic Light Scattering (DLS) instrument. Fluorescence measurements were carried out by fluorescence spectrophotometer. The T1 relaxation time was determined by 0.5 T Bruker minispec relaxometer.

### I. Method of Making Ultra-Small Chitosan Nanoparticles

A stock solution of 0.25% chitosan in 100 ml of 1% acetic acid was prepared. Chitosan was crosslinked with a dicarboxylic acid (tartaric acid) using carbodiimide chemistry in a water-in-oil microemulsion to form the covalently crosslinked chitosan nanoparticles [1], The concentration of dicarboxylic acid taken is 25% of chitosan concentration [2]. The experimental method has two microemulsions, ME-I and ME-II. ME-I comprises cyclohexane (11 mL), n-hexanol (4 mL), chitosan stock solution (4 mL) and Triton-X 100 (6 mL). ME-II comprises of cyclohexane (11 mL), n-hexanol (4 mL), aqueous solution of a mixture of tartaric acid, EDC and n-hydroxysuccinimide (NHS) (4 mL) and Triton X-100 (6 mL). ME-II was added to ME-I, preferably drop by drop, and allowed to react for 24 hours. The chitosan nanoparticles were recovered by adding ethanol to the microemulsion. The nanoparticles were washed with ethanol 4-5 times. The nanoparticles were then dispersed in water followed by dialysis against water for 48 hours. The nanoparticle solution was passed through a 0.2 μm syringe filter. A similar protocol was followed to prepare chitosan nanoparticles for a stock solution of 0.5% chitosan in 100 ml of 1% acetic acid, as set forth below.

For a stock solution of 0.5% chitosan in 100 ml of 1% acetic acid, chitosan was crosslinked with a dicarboxylic acid (tartaric acid) using carbodiimide chemistry in a water-in-oil microemulsion to form the covalently crosslinked chitosan nanoparticles. The concentration of dicarboxylic acid taken is 25% of the chitosan concentration. The experimental method has two microemulsions, ME-I and ME-II. ME-I comprises of cyclohexane (11 mL), n-hexanol (6 mL), chitosan stock solution (4 mL) and Triton-X 100 (8 mL). M.E. II comprises of cyclohexane (11 mL), n-hexanol (6 mL), aqueous solution of a mixture of tartaric acid, EDC and n-hydroxysuccinimide (NHS) (4 mL) and Triton X-100 (8 mL). ME-II was added to ME-I, preferably drop by drop, and allowed to react for 24 hours. The chitosan nanoparticles were recovered by adding ethanol to the microemulsion. The nanoparticles were washed with ethanol 4-5 times. The nanoparticles were then dispersed in water followed by dialysis against water for 48 hours. The nanoparticle solution was passed through a 0.2 μm syringe filter.

### Characterizaton

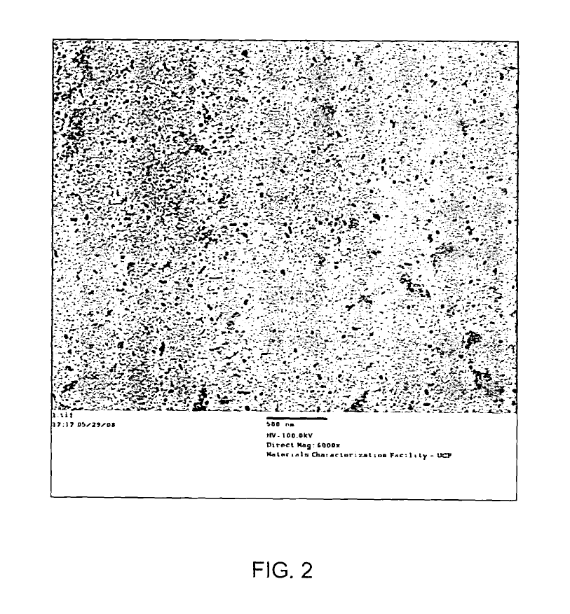

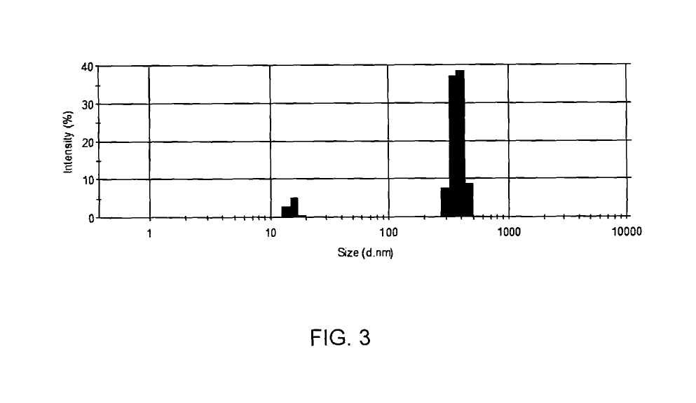

The particle size range of the nanoparticles prepared from 0.25% chitosan (FIG. 2) and 0.50% chitosan was determined by TEM to be approximately 15-20 nm. The representative TEM image of the nanoparticles is presented in FIG. 2. The representative particle size distribution is shown in FIG. 3. The particle size data show two different ranges of distribution, one range at about 10-20 nm and the other above 100 nm which is due to the aggregation of chitosan nanoparticles. The zeta potential of the nanoparticles prepared from 0.25% chitosan is +27 mV and that from 0.50% chitosan solution is +32.8 mV.

### II. Method of Making Ultra Small Fluorescent Chitosan Nanoparticles

A stock solution of 0.25% chitosan in 100 ml of 1% acetic acid was prepared. The chitosan polymer was covalently attached to the fluorescent dye fluorescein isothiocyanate (FITC) [3]. Other fluorescent moieties such as near infrared dye, quantum dots may also be covalently attached to the chitosan polymer and could be employed as alternatives to FITC. The skilled will know fluorescent tags useful in bioimaging for use in the disclosed invention. To 4 mL of the chitosan solution, FITC dissolved in ethanol was added and allowed to stir overnight in dark conditions at room temperature. The FITC labeled chitosan polymer was dialyzed against water for 48 hours. The fluorescent chitosan nanoparticles were prepared as described in Section I. In ME-I, cyclohexane (11 mL), n-hexanol (4 mL), chitosan stock solution (2 mL), FITC labeled chitosan polymer (2 mL) and Triton X-100 (6 mL). ME-II comprises of cyclohexane (11 mL), n-hexanol (4 mL), aqueous solution of a mixture of tartaric acid, EDC and n-hydroxysuccinimide (NHS) (4 mL), Triton X-100 (6 mL). ME-II was added to ME-I and allowed to react for 24 hours. The chitosan nanoparticles were recovered by adding ethanol to the microemulsion. The nanoparticles were washed with ethanol 4-5 times. The nanoparticles were dispersed in water followed by dialysis against water for 48 hours. The nanoparticle solution was passed through a 0.2 μm syringe filter. A similar protocol was followed to prepare chitosan nanoparticles for a stock solution of 0.5% chitosan dissolved in 100 ml of 1% acetic acid.

### Characterization

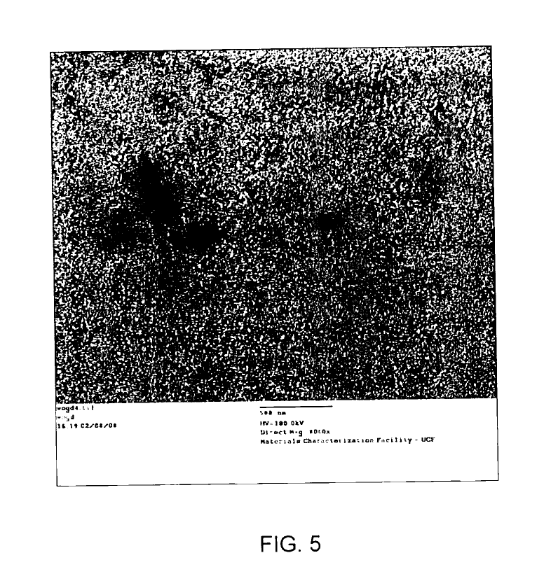

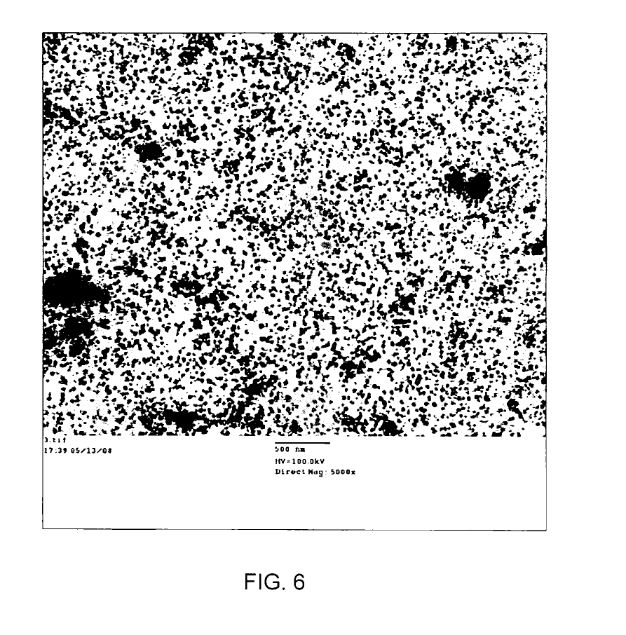

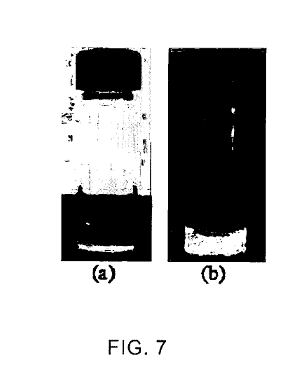

The fluorescent nanoparticles have excitation and emission at 490 nm and 517 nm, respectively, that are characteristic of an FITC moiety. FIG. 5 shows the excitation and emission spectra of FITC in the chitosan nanoparticles. The particle size of the nanoparticles as determined by TEM is 15-20 nm (FIGS. 6 and 7). The zeta potential of the nanoparticles is +24 mV and +29 mV for the particles prepared from 0.25% chitosan and 0.5% chitosan solution, respectively.

### III. Method of Making Paramagnetic Chitosan Nanoparticles

A stock solution of 0.25% chitosan in 100 ml of 1% acetic acid was prepared. The paramagnetic chitosan polymer was prepared by reacting chitosan with a macrocycle such as DOTA to chelate paramagnetic ions like gadolinium, dysprosium, europium etc. To 4 mL of the chitosan solution, DOTA-NHS was added such that the concentration of DOTA-NHS to chitosan is 1:1, 1:3, 1:5 or 1:7. The DOTA-NHS covalently bound to chitosan was then chelated to gadolinium ion by the addition of excess gadolinium acetate hydrate. The excess gadolinium ions are removed by reacting with ethylene diamine tetraacetate disodium salt. The paramagnetic chitosan polymer was dialyzed against water for 48 hours. Presence of gadolinium ion in the chitosan polymer was determined by measuring the T1 relaxation time. Paramagnetic chitosan nanoparticles were prepared as described in Method section II, above.

### IV. Method of Making Radio-Opaque Chitosan Nanoparticles

Radio-opaque contrast agent iohexyl can be incorporated into chitosan nanoparticles as described in Section I.

### V. Methods of Making Bimodal Chitosan Nanoparticles for Bioimaging

Fluorescent and Paramagnetic Chitosan Nanoparticles

The fluorescent and paramagnetic chitosan polymer were prepared as described in Section II and III respectively. In ME-I, cyclohexane (11 mL), n-hexanol (4 mL), FITC labeled chitosan polymer (1.2 mL), paramagnetic chitosan polymer (1.8 mL), chitosan stock solution (1 mL), and Triton-X 100 (6 mL). ME-II comprises of cyclohexane (11 mL), n-hexanol (4 mL), aqueous solution of a mixture of tartaric acid, EDC and n-hydroxysuccinimide (NHS) (4 mL), Triton X-100 (6 mL). ME-II was added to ME-I and allowed to react for 24 hours. The chitosan nanoparticles were recovered by adding ethanol to the microemulsion. The nanoparticles were washed with ethanol 4-5 times. The nanoparticles were dispersed in water followed by dialysis against water for 48 hours. The nanoparticle solution was passed through a 0.2 μm syringe filter. This protocol was carried out with both 0.25% and 0.5% chitosan polymer solutions.

### Characterization:

The particle size of the nanoparticles as determined by TEM is approximately from 15-20 nm. The fluorescent nanoparticles have excitation and emission at 490 nm and 517 nm, respectively, that are characteristic of the FITC moiety. T1 relaxation time was 140 ms and 101 ms for the nanoparticles prepared from 0.25% and 0.5% chitosan solution, respectively, as measured in a 0.5 T relaxometer. The relaxation time for water is 2500 ms. The zeta potential of the nanoparticles is +24 mV and +33 mV.

Fluorescent and Radio-Opaque Chitosan Nanoparticles

The fluorescent chitosan polymer can be prepared as described in Section II. The radio-opaque chitosan polymer can be prepared as described in Section IV. The fluorescent and radio-opaque chitosan nanoparticles can be prepared as described in Section V.

### VI. Method of Making Multifunctional Chitosan Nanoparticles

Target-Specific Fluorescent Chitosan Nanoparticles, Useful in Targeting and Imaging

A stock solution of 0.25% chitosan in 100 ml of 1% acetic acid was prepared. Chitosan nanoparticles can be made target specific to biological entities such as tumor cells, antibodies, etc., by conjugating the appropriate target-specific ligand such as folic acid, antibody, antigen, aptamer, peptide, oligonucleotides, etc. For example, to make folate-conjugated chitosan nanoparticles, first folic acid was attached to chitosan polymer by using EDC followed by dialysis against water. Fluorescent chitosan polymer was prepared as described in Section II. The chitosan nanoparticles that are fluorescent as well as target specific were prepared in a similar method described in Section I. In ME-I, cyclohexane (11 mL), n-hexanol (4 mL), FITC labeled chitosan polymer (1 mL), folate conjugated chitosan polymer (1 mL), chitosan stock solution (1 mL), and Triton-X 100 (6 mL). ME-II comprises cyclohexane (11 mL), n-hexanol (4 mL), aqueous solution of a mixture of tartaric acid, EDC and n-hydroxysuccinimide (NHS) (4 mL) and Triton X-100 (6 mL). ME-II was added to ME-I and allowed to react for 24 hours. The chitosan nanoparticles were recovered by adding ethanol to the microemulsion. The nanoparticles were washed with ethanol 4-5 times. The nanoparticles were dispersed in water, followed by dialysis against water for 48 hours. The nanoparticle solution was passed through a 0.2 μm syringe filter.

### Characterization

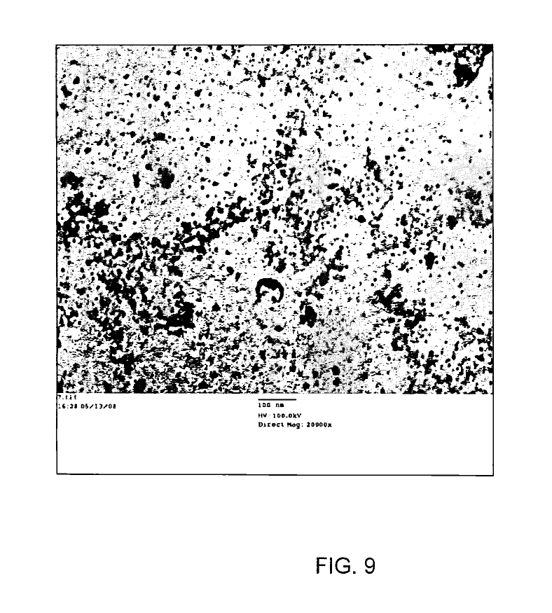

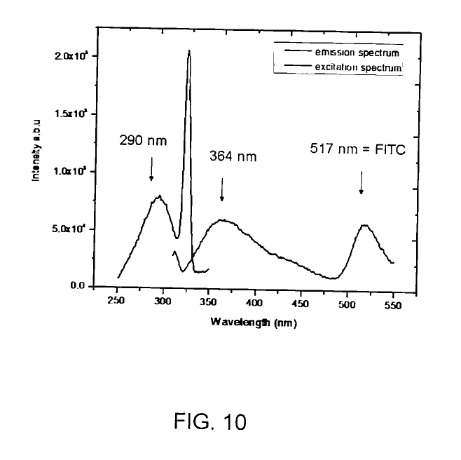

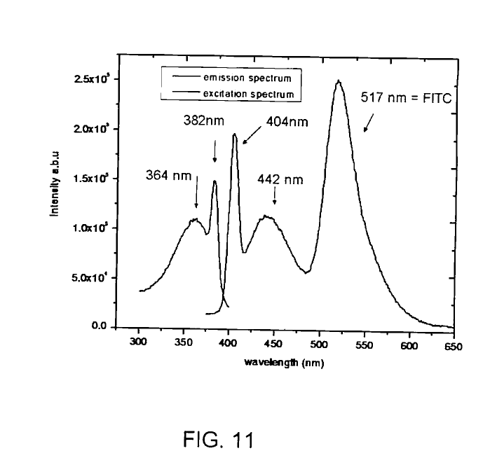

The particle size of the nanoparticles as determined by TEM is 15-20 nm; see FIG. 9. The presence of folate is confirmed from the excitation and emission spectra (shown in FIGS. 10 and 11). When the solution is excited at 290 nm, emission is observed at 364 nm due to the p-aminobenzoic unit of folic acid and when excited at 364 nm, emission is observed at 442 nm due to the methyl pteridine moiety of the folic acid. Presence of FITC was confirmed by excitation wavelength at 490 nm and emission at 517 nm. The zeta potential for the nanoparticles is +22.2 mV.

Drug Loaded Fluorescent Chitosan Nanoparticles fpr Imaging and Drug Delivery

Chitosan nanoparticles that are fluorescently labeled can also be loaded with drugs for applications as a drug delivery vehicle. The presence of the fluorescent tag will help in imaging or, in other words, help in tracking the release of drugs. A stock solution of 0.25% chitosan in 100 ml of 1% acetic acid can be prepared. Fluorescent chitosan polymer can be prepared as described in Section II. The drugs can be added to the ME-I along with the chitosan solution and the nanoparticles can be prepared as described in Method section II. The drugs can be physically attached to the chitosan polymer or can be chemically attached for example by sulfide bonds.

### VII. Method of Making Non-Crosslinked Chitosan Nanoparticles

A stock solution of 0.25% chitosan in 100 ml of 1% acetic acid can be prepared as noted above. Chitosan nanoparticles can be prepared by mixing together a chitosan solution and a modified chitosan solution containing succinic acid functional group. The nanoparticles formed would be held together by electrostatic attraction. The modified chitosan containing succinic acid is prepared by reacting succinic anhydride with chitosan from stock solution for about 24 hours with addition of methanol solvent [4]. Th polymer is precipitated by raising the pH of the solution to 8-10. The precipitate dispersed in water is dialyzed against water. The chitosan solution will be positively charged due to the protonated amine groups and the succinic anhydride chitosan will have an excess of negative charge due to the carboxyl groups. Combining the positively and negatively charged chitosan polymers can result in electrostatically held chitosan nanoparticles.

### VIII. Method to Make Chitosan Nanoparticle Sensors

Chitosan nanoparticles can be prepared as described in Section I for cadmium sensing application similar to the cadmium sensors reported by our group [5].

Accordingly, in the drawings and specification there have been disclosed typical preferred embodiments of the invention and although specific terms may have been employed, the terms are used in a descriptive sense only and not for purposes of limitation. The invention has been described in considerable detail with specific reference to these illustrated embodiments. It will be apparent, however, that various modifications and changes can be made within the spirit and scope of the invention as described in the foregoing specification and as defined in the appended claims.

### ACKNOWLEDGMENTS

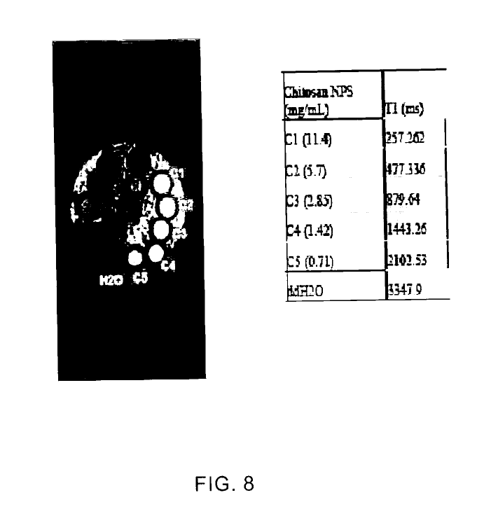

The inventors wish to acknowledge the assistance of the following colleagues. Dr. Soumitra Kar of the Advanced Materials Processing and Analysis Center (AMPAC) of the University of Central Florida, helped us to record TEM images and is sincerely acknowledged for his time. Dr. Glenn A. Walter and his team at the Advanced Magnetic Resonance Imaging and Spectroscopy (AMRIS) Facility, University of Florida, assisted with MRI characterization, as shown in FIG. 8. Dr. Sudipta Seal of the University of Central Florida, allowed us to use his Malvern Zetasizer (Nano ZS) for particle size and surface charge characterization and we appreciate his kindness. The inventors further wish to acknowledge the assistance of the University of Central Florida NanoScience Technology Center for help in characterizing the ultra-small nanoparticles subject of this invention.

### REFERENCES CITED

1. Zhi, J., Wang Y. J. and Luo, G. S. Adsorption of diuretic furosemide onto chitosan nanoparticles prepared with a water-in-oil nanoemulsion system. Reactive and Functional Polymers, 65, 249-257 (2005).
2. Bodnar, M., Hartmann, J. F. and Bobely J. Preparation and characterization of chitosan based nanoparticles. Biomacromolecules 6, 2521-2527, (2005).
3. Huang, M., Ma, Z., Khor, E., and Lim, L. Y. Uptake of FITC-chitosan nanoparticles by A549 cells. Pharmaceutical Research, Vol. 19, 1488-1494, (2002).
4. Rekha, M. R. and Sharma, C. P. pH Sensitive Succinyl Chitosan Microparticles: A Preliminary Investigation Towards Oral Insulin Delivery. Trends in Biomaterials and Artificial Organs. 21, 107-115, (2008).
5. Banerjee, S., Kar, S, and Santra, S. A simple strategy for quantum dot assisted selective detection of cadmium ions. Chemical Communications, 25, 3037, (2008).

That which is claimed:

1. A method of making ultra-small chitosan nanoparticles, the method comprising:
preparing a first microemulsion containing effective amounts of cyclohexane, n-hexanol, chitosan polymer and a nonionic surfactant;
preparing a second microemulsion containing effective amounts of cyclohexane, n-hexanol, tartaric acid, EDC, n-hydroxysuccinimide, and a nonionic surfactant;
reacting the first and second microemulsions for a time sufficient to form the ultra-small chitosan nanoparticles; and
recovering ultra-small nanoparticles from the reacted microemulsion, the ultra-small nanoparticles having a size range of approximately from 10 to 20 nm.

2. The method of claim 1, wherein the chitosan polymer is covalently crosslinked by reacting with a dicarboxylic acid in a water-in-oil microemulsion.

3. The method of claim 1, wherein the chitosan polymer comprises a proportion of the polymer linked to a succinic acid functional group so that recovered nanoparticles are formed by non-crosslinked electrostatically held chitosan and succinic anhydride chitosan.

4. The method of claim 1, wherein the nonionic surfactant comprises Triton X-100.

5. The method of claim 1, wherein the tartaric acid is in an aqueous solution.

6. The method of claim 1, wherein reacting comprises mixing.

7. The method of claim 1, wherein reacting comprises continuous mixing.

8. The method of claim 1, wherein reacting continues for approximately 24 hours.

9. The method of claim 1, wherein recovering is effected by addition of ethanol so as to separate the nanoparticles from the microemulsion.

10. The method of claim 1, further comprising washing the recovered nanoparticles in ethanol at least once.

11. The method of claim 1, further comprising suspending the recovered nanoparticles in a fluid carrier.

12. The method of claim 1, further comprising suspending the recovered nanoparticles in water.

13. The method of claim 1, further comprising suspending the recovered nanoparticles in water and dialysing the suspended nanoparticles against water.

14. The method of claim 1, further comprising suspending recovered nanoparticles in a fluid carrier and separating aggregated nanoparticles from monodispersed nanoparticles after suspending.

15. The method of claim 1, further comprising suspending recovered nanoparticles in a fluid carrier and separating aggregated nanoparticles from monodispersed nanoparticles after suspending by filtration.

16. The method of claim 1, wherein the chitosan polymer is further covalently labeled with a fluorescent tag so that the recovered nanoparticles exhibit fluorescence.

17. The method of claim 16, wherein the fluorescent tag is selected from fluorescein isothiocyanate, a near-infrared dye; a quantum dot, and combinations thereof.

18. The method of claim 1, wherein the chitosan polymer is further linked to a sequestering agent having an MRI contrast agent bound therein so that the recovered nanoparticles are effective as an MRI contrast medium.

19. The method of claim 18, wherein the MRI contrast agent comprises a paramagnetic ion selected from gadolinium, dysprosium, europium, and compounds and combinations thereof.

20. The method of claim 1, wherein the chitosan polymer is further linked with iohexyl so that the recovered nanoparticles are radio-opaque.

21. The method of claim 1, wherein the chitosan polymer comprises a mixture of fluorescent-labeled chitosan and chitosan linked with a sequestering agent having a paramagnetic chelate bound therein so that the recovered nanoparticles are effective as a bimodal agent which is fluorescent as well as paramagnetic.

22. The method of claim 1, wherein the chitosan polymer comprises a mixture of fluorescent-labeled chitosan and chitosan polymer linked with iohexyl so that the recovered nanoparticles are effective as a bimodal agent which is fluorescent as well as radio-opaque.

23. The method of claim 1, wherein the chitosan polymer is conjugated with a ligand for a predetermined biological target so that recovered nanoparticles are effective as target-specific probes.

24. The method of claim 23, wherein the ligand is selected from a peptide, an oligonucleotide, folic acid, an antigen, an antibody, and combinations thereof.

25. The method of claim 1, wherein the chitosan polymer is conjugated with a drug.

26. Nanoparticles comprising chitosan polymer, having a range of from approximately 10 to 20 nm in size and having a zeta potential of approximately +22 to +33 mV.

27. The nanoparticles of claim 26, wherein the chitosan polymer is covalently crosslinked.

28. The nanoparticles of claim 26, wherein the chitosan polymer comprises a proportion of the polymer linked to a succinic acid functional group so that recovered nanoparticles are formed by non-crosslinked electrostatically held chitosan and succinic anhydride chitosan.

29. The nanoparticles of claim 26, wherein the chitosan polymer is covalently labeled with a fluorescent tag so that the nanoparticles exhibit fluorescence.

30. The nanoparticles of claim 26, wherein the chitosan polymer is further linked to a sequestering agent having an MRI contrast agent bound therein so that the nanoparticles are effective as an MRI contrast medium.

31. The nanoparticles of claim 26, wherein the chitosan polymer is further linked with iohexyl so that the nanoparticles are radio-opaque.

32. The nanoparticles of claim 26, wherein the chitosan polymer comprises a mixture of fluorescent-labeled chitosan and chitosan linked with a sequestering agent having a paramagnetic chelate bound therein so that the nanoparticles are effective as a bimodal agent which is fluorescent as well as paramagnetic.

33. The nanoparticles of claim 26, wherein the chitosan polymer comprises a mixture of fluorescent-labeled chitosan and chitosan polymer linked with iohexyl so that the recovered nanoparticles are effective as a bimodal agent which is fluorescent as well as radio-opaque.

34. The nanoparticles of claim 26, wherein the chitosan polymer is conjugated with a ligand for a predetermined biological target so that nanoparticles are effective as target-specific probes.

35. The nanoparticles of claim 26, wherein the chitosan polymer is conjugated with a biologically active drug.
